(19) United States
(12) Patent Application Publication
(10) Pub. No.: US 2026/0315598 A1
(43) Pub. Date: Oct. 15, 2026

(54) PHOTOSENSITIZER CHLORIN E6 (Ce6)-LOADED NANOPARTICLE TARGETING MITOCHONDRIA OF BREAST CANCER CELL, AND PREPARATION METHOD AND USE THEREOF

(71) Applicant: QILU UNIVERSITY OF TECHNOLOGY(SHANDONG ACADEMY OF SCIENCES), Jinan (CN)

(72) Inventors: Yingshu Guo, Jinan (CN); Dan Jing, Jinan (CN)

(73) Assignee: QILU UNIVERSITY OF TECHNOLOGY(SHANDONG ACADEMY OF SCIENCES), Jinan (CN)

(21) Appl. No.: 18/836,185

(22) PCT Filed: Feb. 24, 2024
(86) PCT No.: PCT/CN2024/075654

§ 371 Date: Aug. 6, 2024

(30) Foreign Application Priority Data
Dec. 12, 2023 (CN) 202311714847.1

(51) Int. Cl. A61K 41/00; A61K 9/51; A61K 47/69; A61P 35/00

(52) U.S. Cl. CPC A61K 41/0071; A61K 9/5115; A61K 9/5176; A61K 47/6949; A61P 35/00

(57) ABSTRACT

The present disclosure provides a photosensitizer chlorin e6 (Ce6)-loaded nanoparticle targeting mitochondria of a breast cancer cell, and a preparation method and use thereof, and belongs to the field of pharmaceutical preparations. In the present disclosure, the nanoparticle uses a hollow mesoporous silica nanoparticle (HMSN) as a carrier, a hollow position of the carrier is loaded with a photosensitizer Ce6, the carrier is externally encapsulated with a red blood cell membrane (RBCM), and the carrier after encapsulating the RBCM is modified with a Pt nanocluster on one side as well as an aptamer LXL-1 and 1-carboxypropyltriphenylphosphine bromide (TPP) on the other side.

### CROSS REFERENCE TO RELATED APPLICATION

The present application is a national stage application of International Patent Application No. PCT/CN2024/075654, filed on Feb. 4, 2024, claims priority to Chinese Patent Application No. 202311714847.1 filed to the China National Intellectual Property Administration (CNIPA) on Dec. 12, 2023 and entitled “PHOTOSENSITIZER CHLORIN E6 (Ce6)-LOADED NANOPARTICLE TARGETING MITOCHONDRIA OF BREAST CANCER CELL, AND PREPARATION METHOD AND USE THEREOF”, both of which are incorporated herein by references in their entities.

### REFERENCE TO SEQUENCE LISTING

A computer readable XML file entitled “GWPCTP20231209901”, that was created on Jul. 2, 2024, with a file size of about 2100 bytes, contains the sequence listing for this application, has been filed with this application, and is hereby incorporated by reference in its entirety.

### TECHNICAL FIELD

The present disclosure belongs to the field of pharmaceutical preparations, and in particular relates to a photosensitizer chlorin e6 (Ce6)-loaded nanoparticle targeting mitochondria of a breast cancer cell, and a preparation method and use thereof.

### BACKGROUND

Breast cancer is a type of malignant tumor in which breast tissue becomes cancerous and continues to grow indefinitely due to factors such as genes and hormone disorders. The breast cancer is the most common cancer in women, and mainly includes ductal carcinoma and lobular carcinoma. The treatment of breast cancer mainly includes surgical resection, chemotherapy, radiotherapy or a combination thereof. However, traditional chemotherapy and radiotherapy methods have serious side effects due to lack of targeting to tumor cells.

Aptamers refer to single-stranded oligonucleotides that are screened from synthetic single-stranded DNA/RNA libraries and can bind to target molecules with high affinity and high specificity. The aptamer relies on intermolecular forces such as hydrogen bonds, Van der Waals' force, and hydrophobic interactions to form special three-dimensional structures, such as hairpins, pseudoknots, convex rings, and G-tetramers, thereby specifically recognizing target substances and affect their biological activity. Aptamer probes developed in recent years, especially cell-targeted aptamers, can specifically identify cancer cells in complex samples including whole blood samples, thus bringing new hope and solutions to early specific and rapid diagnosis and targeted treatment of tumors. Nanoparticle drug delivery systems can improve the delivery efficiency of anti-cancer drugs and reduce drug side effects. These systems mainly prevent non-targeted tissues or parts from being exposed to therapeutic drugs or causing damage to targeted tissues due to high doses of specific drugs.

Chlorin e6 (Ce6), as a widely used photosensitizer, has a molecular weight of 596.67 KD and a molecular formula of C₃₄H₃₆N₄O₆, and belongs to a member of the chlorophyll family. On one hand, the Ce6 is an amphiphilic (water-soluble and fat-soluble) molecule with absorption peaks around 400 nm and 660 nm. Under the 660 nm laser, this molecule shows stronger tissue penetration than that of some traditional photosensitizers such as photoporphyrin. On the other hand, the Ce6 exhibits an extremely high singlet oxygen quantum yield and has desirable applications in photodynamic therapy. However, due to the amphipathic nature, it is difficult to wrap Ce6 into stable nanoparticles using common nanoparticle preparation methods. For example, it is difficult to coat the Ce6 with a common carrier albumin using common methods. Therefore, it is one of the keys to research in this field to prepare a Ce6-coated nanoparticle drug delivery system and combine same with a targeting aptamer.

### SUMMARY

In view of this, an objective of the present disclosure is to provide a photosensitizer Ce6-loaded nanoparticle targeting mitochondria of a breast cancer cell. The nanoparticle shows targeting properties, and can enter the mitochondria of breast cancer cells MDA-MB-231 and generate ¹O₂ under irradiation of 808 nm laser to damage the mitochondria and release DNA, thereby causing cell death.

To achieve the above objective, the present disclosure provides the following technical solutions:

The present disclosure provides a photosensitizer Ce6-loaded nanoparticle targeting mitochondria of a breast cancer cell, where the nanoparticle uses a hollow mesoporous silica nanoparticle (HMSN) as a carrier, a hollow position of the carrier is loaded with a photosensitizer Ce6, the carrier is externally encapsulated with a red blood cell membrane (RBCM), and the carrier after encapsulating the RBCM is modified with a Pt nanocluster on one side as well as an aptamer LXL-1 and 1-carboxypropyltriphenylphosphine bromide (TPP) on the other side.

Preferably, the RBCM is a mouse RBCM.

Preferably, the aptamer LXL-1 has a nucleotide sequence set forth in SEQ ID NO: 1.

Preferably, a 5′-end of the aptamer LXL-1 is modified with a carboxyl group (—COOH).

The present disclosure further provides a preparation method of the nanoparticle, including the following steps:
(1) subjecting the HMSN to ultrasonic mixing with a dimethyl sulfoxide (DMSO) solution of Ce6 to allow freeze-drying to obtain a Ce6-loaded HMSN nanoparticle (denoted as an HC nanoparticle);
(2) mixing a phosphate-buffered saline (PBS) solution of the HC nanoparticle with a PBS solution of an RBCM powder to allow filter membrane extrusion to obtain an RBCM-coated HC nanoparticle (denoted as an RHC nanoparticle);
(3) dispersing the RHC nanoparticle in the PBS solution, conducting centrifugation, spin-coating a resulting precipitate to a surface of poly-L-lysine, conducting incubation at room-temperature, washing with the PBS solution, and removing a resulting supernatant; adding a positively-charged platinum (Pt) nanocluster solution into a remaining substance, conducting incubation at the room-temperature, washing with the PBS solution, and removing a resulting supernatant to obtain an asymmetrically-modified nanoparticle (denoted as a Pt@RHC nanoparticle); and
(4) dispersing the Pt@RHC nanoparticle in a mixed PBS solution containing LXL-1, TPP—COOH, 1-ethyl-3-(3-dimethylaminopropyl) carbodiimide hydrochloride (EDC), and N-hydroxysulfosuccinimide sodium salt (NHS) to allow a reaction, stirring at a room temperature, conducting centrifugation, washing a resulting precipitate with the PBS solution, and re-dispersing the precipitate in the PBS solution to obtain a modified nanoparticle (denoted as a Pt@RHC-LXL-1/TPP nanoparticle).

Preferably, a method for preparing the HMSN in step (1) includes the following steps: adding solid silica into a high-purity aqueous solution of cetyltrimethylammonium chloride (CTAC) and triethanolamine (TEA) to obtain a mixed solution; adding a tetraethyl orthosilicate (TEOS) solution into the mixed solution and stirring in a water bath to form a silica surface-coated silica nanoparticle; and etching the silica surface-coated silica nanoparticle to obtain the HMSN.

Preferably, the solid silica is prepared by mixing absolute ethanol, water, and ammonia water, stirring a resulting mixture at a room temperature, and then adding the TEOS solution into the mixture.

Preferably, the absolute ethanol, the water, the ammonia water, and the TEOS solution are at a volume ratio of 35-36:4-6:1-1.2:1, and the TEOS solution has a mass fraction of 98%.

Preferably, the stirring at the room temperature is conducted for 5 min to 10 min, and a reaction is conducted for 50 min to 70 min.

Preferably, in each 30 mL of the high-purity aqueous solution, a mass ratio of the CTAC, the TEA, and the solid silica is 2 g:20 mg:100 mg; and the mixed solution and the TEOS solution are at a volume ratio of 30:0.15, while the TEOS solution has a mass fraction of 98%.

Preferably, the stirring in the water bath is conducted at 75° C. to 85° C. for 50 min to 70 min.

Preferably, the etching includes the following steps: cooling the silica surface-coated silica nanoparticle to 45° C. to 50° C. in a water bath, adding Na₂CO₃, and stirring continuously to obtain the HMSN.

Preferably, the obtained HMSN is washed with a mixed cleaning solution, and the mixed cleaning solution is obtained by mixing absolute ethanol with concentrated hydrochloric acid at a volume ratio of 1:10.

Preferably, the HMSN and the Ce6 are at a mass ratio of 4-5:0.1-0.2, and the ultrasonic mixing is conducted for 8 min to 12 min at an ultrasonic frequency of 40 KHz in step (1).

Preferably, the HC nanoparticle is washed with ultrapure water.

Preferably, the HC nanoparticle and the RBCM powder in step (2) are at a mass ratio of 1:1.

Preferably, a method for preparing the RBCM powder includes the following steps: adding water into a red blood cell suspension, allowing to stand such that red blood cells absorb water and burst, conducting centrifugation to remove hemoglobin, washing and dispersing a resulting precipitate with a PBS solution, and freeze-drying to obtain the RBCM powder.

Preferably, the centrifugation is conducted at 14,000 rpm for 10 min.

Preferably, the filter membrane extrusion in step (2) includes: conducting extrusion with 450 nm and 200 nm filter membranes in sequence, with each extrusion 4 to 5 times.

Preferably, the centrifugation is conducted at 900 rpm for 3 min and the incubation is conducted for 50 min to 70 min in step (3).

Preferably, a method for preparing the positively-charged Pt nanocluster solution includes the following steps: adding an H₂PtCl₆ aqueous solution into ultrapure water, adding a protamine aqueous solution, then conducting violent stirring at a room temperature for 30 min, adding NaBH₄ dropwise, stirring a resulting mixture continuously at a room temperature, and conducting dialysis for 24 h to obtain the positively-charged Pt nanocluster solution.

Preferably, the H₂PtCl₆ aqueous solution has a concentration of 16 mM, the protamine aqueous solution has a concentration of 5 mg/mL, and the NaBH₄ has a concentration of 500 mM; and the H₂PtCl₆ aqueous solution, the ultrapure water, the protamine aqueous solution, and the NaBH₄ are at a volume ratio of 0.8:40:0.5:0.4.

Preferably, the centrifugation is conducted at 900 rpm for 3 min and the incubation is conducted for 50 min to 70 min in step (3).

Preferably, the TPP—COOH has a concentration of 0.05 g/L to 0.15 g/L, the EDC has a concentration of 0.7 g/L to 0.9 g/L, the NHS has a concentration of 0.3 g/L to 0.5 g/L, and the LXL-1 has a concentration of 100 nM in step (4).

Preferably, the reaction is conducted for 12 min to 18 min, the stirring at the room temperature is conducted for 11 h to 13 h, and the centrifugation is conducted at 12,000 rpm for 15 min in step (4).

The present disclosure further provides use of the nanoparticle or the preparation method of the nanoparticle in preparation of a drug for treating breast cancer.

The present disclosure further provides use of the nanoparticle or the preparation method of the nanoparticle in treatment of breast cancer, where the nanoparticle is combined with an 808 nm laser.

Compared with the prior art, the present disclosure has the following beneficial effects:

In the present disclosure, the nanoparticle can successfully enter the cells through the special targeting of the aptamer LXL-1 to breast cancer cells MDA-MB-231. By using the Pt nanocluster as a power device of the nanoparticle, it overcomes a Brownian force in the environment and catalyzes H₂O₂ to produce oxygen in the tumor tissue environment, alleviating a hypoxic environment of tumor cells. By targeting mitochondria through the TPP under the action of light conditions and intracellular mechanisms, the photosensitizer Ce6 loaded in the hollow position of the carrier leaks into the mitochondria and cytoplasm. At this time, ¹O₂ is produced under light conditions to damage mitochondria and release DNA into the cytoplasm, causing cell damage and ultimately cell death.

### BRIEF DESCRIPTION OF THE DRAWINGS

FIGS. 1A-1B shows a transmission electron microscopy (TEM) image and a dynamic light scattering (DLS) data of the solid silica (dSiO₂), where FIG. 1A is the TEM image and FIG. 1B is the DLS data;

FIGS. 2A-2B shows a TEM image and a DLS data of the HMSN, where FIG. 2A is the TEM image and FIG. 2B is the DLS data;

FIGS. 3A-3B shows a nitrogen adsorption-desorption isotherm and pore diameter distribution of the HMSN, where FIG. 3A is the nitrogen adsorption-desorption isotherm and FIG. 3B is the pore diameter distribution;

FIGS. 4A-4B shows a TEM image and a DLS data of the positively-charged Pt nanoclusters (PtNPs), where FIG. 4A is the TEM image and FIG. 4B is the DLS data;

FIG. 5 shows the zeta potentials of HMSN, HC, RBCM, PtNPs, RHC, Pt@RHC, LXL-1, and Pt@RHC-LXL-1/TPP;

FIGS. 6A-6B shows an ultraviolet absorption peak and a fluorescence spectrum intensity of the nanoparticle, where FIG. 6A is the ultraviolet absorption curve of HMSN, Ce6, and HC, and FIG. 6B is the fluorescence intensity of Ce6 and HC;

FIGS. 7A-7D shows the ability of Pt@RHC-LXL-1/TPP in producing ¹O₂ under light conditions, where FIG. 7A is the fluorescence curve over time after laser irradiation; FIG. 7B is the UV absorption curve over time after laser irradiation; FIG. 7C is the UV absorption curve after laser irradiation for 10 min after incubation with different H₂O₂ concentrations; and FIG. 7D is the UV absorption curve after laser irradiation for 10 min after incubation with Pt@RHC-LXL-1/TPP nanoparticles at different concentrations;

FIGS. 8A-8F shows an influence of the nanoparticle on a cell viability of the MDA-MB-231 cells, where FIG. 8A to FIG. 8E are the cell viabilities of MDA-MB-231 cells after co-incubation with HMSN, Pt@RHC, Pt@RHC-LXL-1, Pt@RHC-LXL-1/TPP, and Pt@RH-LXL-1/TPP with treatment of illumination, respectively; and FIG. 8F is the cell viabilities of MDA-MB-231 cells after co-incubation with Pt@RHC-LXL-1/TPP without illumination;

FIGS. 9A-9B shows targeting results of the Pt@RH-LXL-1/TPP nanoparticle under a fluorescence microscope, where FIG. 9A is the fluorescence images of breast cancer cells MCF-7 after incubation with the Pt@RH-LXL-1/TPP nanoparticle for 1 h, 2 h, and 3 h, respectively; and FIG. 9B is the fluorescence images of breast cancer cells MDA-MB-231 after incubation with the Pt@RH-LXL-1/TPP nanoparticle for 1 h, 2 h, and 3 h, respectively; scale bars: 50 μm; and

FIG. 10 shows the intracellular production of ¹O₂ after co-incubation of different nanoparticles with the breast cancer cells MDA-MB-231; scale bars: 20 μm.

### DETAILED DESCRIPTION OF THE EMBODIMENTS

The present disclosure provides a photosensitizer Ce6-loaded nanoparticle (denoted as Pt@RHC-LXL-1/TPP) targeting mitochondria of a breast cancer cell, where the nanoparticle uses an HMSN as a carrier, a hollow position of the carrier is loaded with a photosensitizer Ce6, the carrier is externally encapsulated with an RBCM, and the carrier after encapsulating the RBCM is modified with a Pt nanocluster on one side as well as an aptamer LXL-1 and TPP on the other side. The breast cancer cell is preferably an breast cancer cell MDA-MB-231.

In the present disclosure, using RBCM to encapsulate the carrier loaded with the photosensitizer Ce6 can increase an existence time of the carrier in vivo, thereby preventing the carrier from being phagocytized by immune cells after entering the body. The Pt nanocluster can asymmetrically modify the nanoparticle and then serve as a power device for the nanoparticle to overcome a Brownian force in the environment. Meanwhile, on the other side of the nanoparticle, the aptamer LXL-1 specifically targeting breast cancer cells MDA-MB-231 and the TPP targeting mitochondria are modified to finally form the nanoparticle Pt@RHC-LXL-1/TPP. This process enhances the targeting of the nanoparticle to breast cancer cells MDA-MB-231 and increases the probability of the nanoparticle entering breast cancer cells MDA-MB-231.

Since the concentration of H₂O₂ in tumor cells is higher than that of normal cells, the Pt nanocluster in the present disclosure can catalyze the H₂O₂ to produce oxygen in the tumor tissue environment, thereby alleviating the hypoxic environment of tumor cells. After entering cells, the TPP targets the mitochondria of cancer cells under the action of light conditions and intracellular mechanisms, the photosensitizer Ce6 loaded in the hollow position of the carrier leaks into the mitochondria and cytoplasm. At this time, ¹O₂ is produced under light conditions to damage mitochondria and release DNA into the cytoplasm, causing cell damage and ultimately cell death.

In the present disclosure, the aptamer LXL-1 has a nucleotide sequence set forth in 5′-GAATTCAGTCGGACAGCGAAGTAGTTTTCCTTCTAACCTAAGAACCCGCGGCAGTT TAATGTAGATGGACGAATACGTCTCCC-3′, as shown in SEQ ID NO: 1. A 5′-end of the aptamer LXL-1 is modified with a carboxyl group (—COOH).

The present disclosure further provides a preparation method of the nanoparticle, including the following steps:
(1) subjecting the HMSN to ultrasonic mixing with a dimethyl sulfoxide (DMSO) solution of Ce6 to allow freeze-drying to obtain a Ce6-loaded HMSN nanoparticle (denoted as an HC nanoparticle);
(2) mixing a PBS solution of the HC nanoparticle with a PBS solution of an RBCM powder to allow filter membrane extrusion to obtain an RBCM-coated HC nanoparticle (denoted as an RHC nanoparticle);
(3) dispersing the RHC nanoparticle in the PBS solution, conducting centrifugation, spin-coating a resulting precipitate to a surface of poly-L-lysine, conducting incubation at room-temperature, washing with the PBS solution, and removing a resulting supernatant; adding a positively-charged Pt nanocluster solution into a remaining substance, conducting the incubation at room-temperature, washing with the PBS solution, and removing a resulting supernatant to obtain an asymmetrically-modified nanoparticle (denoted as a Pt@RHC nanoparticle); and
(4) dispersing the Pt@RHC nanoparticle in a PBS solution containing LXL-1, TPP—COOH, EDC, and NHS to allow a reaction, stirring at a room temperature, conducting centrifugation, washing a resulting precipitate with the PBS solution, and re-dispersing the precipitate in the PBS solution to obtain a modified nanoparticle Pt@RHC-LXL-1/TPP.

In the present disclosure, the HMSN and the Ce6 are at a mass ratio of 4-5:0.1-0.2, preferably 5:0.133, and the ultrasonic mixing is conducted for 8 min to 12 min, preferably 9 min to 11 min, more preferably 10 min at an ultrasonic frequency of preferably 40 KHz in step (1). Preferably, ultrapure water washing is conducted after the freeze-drying to obtain the nanoparticle HC of high purity.

Alternatively, a method for preparing HMSN includes: 1, mixing absolute ethanol, water, and ammonia water, stirring at a room temperature, then adding a TEOS solution to obtain a mixture, and conducting reaction of the obtained mixture at a room temperature to obtain solid silica (dSiO₂). 2, dissolving CTAC and TEA in high-purity water, stirring at a room temperature, adding a dSiO₂ aqueous solution, stirring at a room temperature, adding TEOS, and stirring a resulting mixture in a water bath to form silica surface-coated silica (dSiO₂@MSN). 3, cooling the dSiO₂@MSN in a water bath, then adding Na₂CO₃ to allow continuous stirring to form the HMSN.

In step 1, the absolute ethanol, the water, the ammonia water, and the TEOS solution are at a volume ratio of preferably 35-36:4-6:1-1.2:1; the stirring is conducted for 5 min to 10 min at room-temperature; the TEOS solution has a mass fraction of 98%; and the reaction is conducted for 50 min to 70 min at room-temperature. In step 2, the CTAC, the TEA, and the dSiO₂ are at a mass ratio of 2 g:20 mg:100 mg, and the TEOS has a volume of 0.15 mL; the water bath is at 75° C. to 85° C.; and the stirring is conducted for 50 min to 70 min. In step 3, the solution is cooled to 45° C. to 50° C., and the HMSN is washed multiple times with a mixed cleaning solution of absolute ethanol and concentrated hydrochloric acid (volume ratio: 1:10) to remove the CTAC attached to a surface of the HMSN.

In step (2), the HC nanoparticle and the RBCM powder are at a mass ratio of 1:1; preferably, the PBS solution of the HC nanoparticle and the PBS solution of the RBCM powder are at a volume ratio of 1:1. Alternatively, the method for preparing the RBCM powder includes: adding water into a red blood cell suspension, allowing to stand such that red blood cells absorb water and burst, conducting centrifugation preferably at 14,000 rpm for 10 min to remove hemoglobin, washing and dispersing a resulting precipitate with a PBS solution, and freeze-drying to obtain the RBCM powder.

In the present disclosure, the filter membrane extrusion in step (2) includes: conducting extrusion with 450 nm and 200 nm filter membranes in sequence, with each extrusion 4 to 5 times.

In the present disclosure, the centrifugation is conducted at 900 rpm for 3 min and the incubation is conducted at room-temperature for 50 min to 70 min, preferably 60 min in step (3). A method for spin-coating the RHC nanoparticle to the surface of the poly-L-lysine includes: coating a solution of the poly-L-lysine (PLL) with a concentration of 0.01% evenly onto a bottom of a 12-well plate at 50 μL per square centimeter, allowing to stand at room temperature for 5 min, and absorbing the excess PLL solution to obtain a PLL-coated 12-well plate. Adding PBS suspension of the RHC nanoparticle into the PLL-coated 12-well plate, and conducting centrifugation at 900 rpm for 3 min such that the nanoparticle is embedded into the PLL on the surface of the 12-well plate under the action of centrifugal force. After removing a resulting supernatant, adding a positively-charged Pt nanocluster solution into the well plate, where the positively-charged Pt nanocluster solution has a concentration of preferably 20 mg/mL, and the nanoparticle and the Pt nanocluster in the final solution are at a mass ratio of 4-6:1, preferably 5:1.

Alternatively, a method for preparing the positively-charged Pt nanocluster includes: adding an H₂PtCl₆ aqueous solution into ultrapure water, adding a protamine aqueous solution, conducting violent stirring at a room temperature for 30 min, adding NaBH₄ dropwise within 30 s to reduce the H₂PtCl₆. Stirring a resulting mixture continuously at a room temperature, and conducting dialysis for 24 h to obtain a crude product, and purifying the obtained crude product to obtain the PtNPs. Preferably, the H₂PtCl₆ aqueous solution has a concentration of 16 mM, the protamine aqueous solution has a concentration of 5 mg/mL, and the NaBH₄ has a concentration of 500 mM; and the H₂PtCl₆ aqueous solution, the ultrapure water, the protamine aqueous solution, and the NaBH₄ are at a volume ratio of 0.8:40:0.5:0.4.

In the present disclosure, the TPP—COOH has a concentration of 0.05 g/L to 0.15 g/L, preferably 0.1 g/L, the EDC has a concentration of 0.7 g/L to 0.9 g/L, preferably 0.8 g/L, the NHS has a concentration of 0.3 g/L to 0.5 g/L, preferably 0.4 g/L, and the LXL-1 has a concentration of 100 nM in step (4). The Pt@RHC nanoparticle and the PBS solution containing LXL-1, TPP—COOH, EDC, and NHS are at a mass-to-volume ratio of 4-6 mg:1 mL, preferably 5 mg:1 mL.

In the present disclosure, the reaction is conducted for 12 min to 18 min, preferably 15 min, the stirring is conducted for 11 h to 13 h, preferably 12 h, and the centrifugation is conducted at 12,000 rpm for 15 min in step (4).

The present disclosure further provides use of the nanoparticle or the method for preparing the nanoparticle in preparation of a drug for treating breast cancer.

The technical solution provided by the present disclosure will be described in detail below with reference to the examples, but they should not be construed as limiting the claimed scope of the present disclosure.

In specific examples of the present disclosure, 1-carboxypropyltriphenylphosphine bromide (TPP), 1,3-diphenylisobenzofuran (DPBF), coumarin, chlorin e6 (Chlorin e6, Ce6), and dimethyl sulfoxide (DMSO) are purchased from Macklin. The dialysis bag and poly-L-lysine (PLL) are purchased from Shanghai Yuanye Biotechnology Co., Ltd. The aptamer LXL-1, protamine, cetyltrimethylammonium chloride (CTAC), N-hydroxysulfosuccinimide sodium salt (NHS), 1-ethyl-3-(3-dimethylaminopropyl) carbodiimide hydrochloride (EDC), triethanolamine (TEA), and MTT cell proliferation and cytotoxicity assay kits are purchased from Sangon Biotech (Shanghai) Co., Ltd. Platinic Chloride (H₂PtCl₆), sodium carbonate, tetraethyl orthosilicate (TEOS), sodium borohydride (NaBH₄), absolute ethanol, and ammonia water are purchased from Sinopharm Group. 2% of ordinary mouse red blood cells are purchased from Shanghai Yuchun Biology Co., Ltd. DAPI, reactive oxygen species detection kit, and RBCM far-infrared fluorescent probe (DiD) are purchased from Beyotime. Fetal bovine serum (FBS) is purchased from Solarbio.

In specific examples of the present disclosure, MDA-MB-231 breast cancer cells are purchased from Wuhan Pricella Biotechnology Co., Ltd. and cultured in a constant-temperature and constant-humidity chamber at 37° C. with a humidity of 95% and a CO₂ concentration of 5%. The medium used for cells is DMEM containing 10% FBS and 1% double antibodies (penicillin-streptomycin).

In the following examples, all methods are conventional methods, unless otherwise specified.

All materials and reagents used in the following examples may be commercially available, unless otherwise specified.

### Example 1

### 1. Preparation of Pt@RHC-LXL-1/TPP Nanoparticles:

In a first step, 35.7 mL of absolute ethanol, 5 mL of water, and 0.8 mL of ammonia water were mixed and stirred at room temperature for 5 min to 10 min. 1 mL of TEOS solution with a mass fraction of 98% was added, and a mixture was reacted at room temperature for 1 h to obtain solid silica (dSiO₂).

In a second step, dSiO₂ nanoparticles were washed 3 times with water and ethanol in sequence, and 200 mg of the dSiO₂ nanoparticles were suspended in 20 ml of water. CTAC (2 g) and TEA (20 mg) were dissolved in 20 mL of high-purity water, stirred at room temperature for 1 h, then added with 10 mL of dSiO₂ aqueous solution, stirred at room temperature for 1 h, and added with 0.15 mL of TEOS. A resulting mixture was stirred in a water bath at 80° C. for 1 h to form silica surface-coated silica, namely dSiO₂@MSN.

In a third step, the dSiO₂@MSN was etched to form HMSN: the mixture obtained in the second step is cooled to 50° C. in a water bath, then added with 636 mg of Na₂CO₃, and stirred for 30 min to form the HMSN. In order to remove CTAC attached to the surface of HMSN, the HMSN was washed multiple times with a mixed cleaning solution of absolute ethanol and concentrated hydrochloric acid (with a volume ratio of 1:10).

In the fourth step, HMSN was coated with photosensitizer Ce6. 133 μL of 1.0 mg/mL Ce6 in DMSO solution was added to 5 mg of the HMSN, subjected to ultrasonic mixing (frequency 40 KHz) for 10 min, freeze-dried, and washed with ultrapure water to obtain HMSN-coated Ce6, namely HC.

In a fifth step, RBCM was prepared: 5 times the volume of deionized water was added to a purchased 2% mouse erythrocyte suspension, mixed well, and allowed to stand at 4° C. for 1 h to make the erythrocytes absorb water and burst. The hemoglobin in the suspension was removed by centrifugation at 14,000 rpm for 10 min, a supernatant was discarded, and a bottom pellet was washed three times with PBS to obtain a pale pink RBCM pellet. The obtained pale pink RBCM pellet was dispersed in PBS solution, stored at −20° C., and then freeze-dried to obtain an RBCM powder, which was stored at −20° C. for subsequent experiments.

In a sixth step, the HC was coated with RBCM: 0.5 mL of HC in PBS solution (2 mg/mL) and 0.5 mL of RBCM in PBS solution (2 mg/mL) were mixed, followed by extrusion with 450 nm and 200 nm filter membranes in sequence, with each extrusion 5 times, to finally obtain RBCM-coated HC, namely RHC.

In a seventh step, a, method for preparing the positively-charged Pt nanocluster included: 40 mL of ultrapure water was added with an H₂PtCl₆ aqueous solution (800 μL, 16 mM), added with a protamine aqueous solution (500 μL, 5 mg/mL), then conducting violent stirring at a room temperature for 30 min, then added with NaBH₄ (400 μL, 500 mM) dropwise within 30 s to reduce the H₂PtCl₆. A resulting mixture was stirred continuously at room temperature for 2 h, and subjected to dialysis for 24 h to obtain a crude product, and the obtained crude product was purified to obtain the PtNPs.

In an eighth step, the RHC was asymmetrically modified with the PtNPs: a solution of the poly-L-lysine (PLL) with a concentration of 0.01% was coated evenly onto a bottom of a 12-well plate at 50 μL per square centimeter, allowed to stand at a room temperature for 5 min, and the excess PLL solution was aspirated to obtain a PLL-coated 12-well plate. The PLL-coated 12-well plate was added with a PBS suspension of the RHC nanoparticle (2 mL, 20 mg/mL), and centrifuged at 900 rpm for 3 min such that the nanoparticle was embedded into the PLL on the surface of the 12-well plate under the action of centrifugal force, incubated at room temperature for 1 h, a resulting supernatant was removed, and the remaining substance was washed three times with PBS to remove unattached nanoparticles. After washing with PBS, the PBS solution was removed, 400 μL of Pt nanocluster solution with a concentration of 20 mg/mL was added into the well, incubated for 1 h, and then washed with PBS solution to obtain an asymmetrically modified nanoparticle Pt@RHC.

In a ninth step, the nanoparticle was modified with aptamer LXL-1 and TPP: 50 mg of the Pt@RHC nanoparticle was dispersed in 10 mL of PBS solution containing LXL-1 (100 nM), TPP—COOH (0.1 g/L), EDC (0.8 g/L), and NHS (0.4 g/L); a resulting mixture was stirred at room temperature for 12 h and centrifuged (12,000 rpm, 15 min); obtained nanoparticle was washed with PBS and re-dispersed in the PBS to obtain Pt@RHC-LXL-1/TPP.

### 2. Preparation of HMSN Nanoparticles:

A preparation method was the same as the first to third steps in the preparation of Pt@RHC-LXL-1/TPP nanoparticles.

### 3. Preparation of Pt@RHC Nanoparticles:

A preparation method was the same as the first to eighth steps in the preparation of Pt@RHC-LXL-1/TPP nanoparticles.

### 4. Preparation of Pt@RHC-LXL-1 Nanoparticles:

A preparation method was the same as the first to eighth steps in the preparation of Pt@RHC-LXL-1/TPP nanoparticles.

In a ninth step, the nanoparticle was modified with aptamer LXL-1 and TPP: 50 mg of the Pt@RHC nanoparticle was added to 10 mL of PBS solution containing LXL-1 (100 μL, 100 nM); a resulting mixture was stirred at room temperature for 12 h and centrifuged (12,000 rpm, 15 min); obtained nanoparticle was washed with PBS and re-dispersed in the PBS to obtain Pt@RHC-LXL-1.

### 5. Preparation of Pt@RH-LXL-1/TPP Nanoparticles:

Compared with the preparation of Pt@RHC-LXL-1/TPP nanoparticles, the fourth step was abandoned (not coating the photosensitizer Ce6), and the HC was replaced with HMSN.

### Example 2

### Detection of the Nanoparticles Prepared in Example 1:

### 1. Characterization of Nanomaterials

Particle size and Zeta potential measurements were tested by a laser particle size analyzer (Zetasizer Nano ZS90) at 25° C. A Nano-ZS90 Zeta potentiometer (Malvern Instrument Ltd., UK) was used to measure the particle size of nanomaterials. The material morphology was characterized by a transmission electron microscope (JEM2100). UV-visible spectra were recorded on a Cary 60 UV-vis-NIR spectrophotometer.

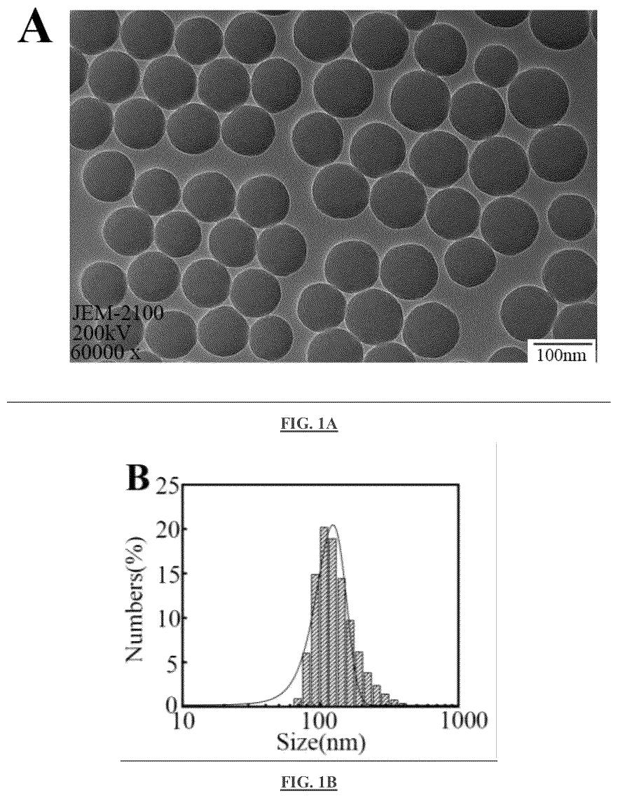

FIGS. 1A-1B shows a TEM image (FIG. 1A) and a DLS data (FIG. 1B) of the solid silica (dSiO₂). In FIG. 1, the TEM data and EDS showed that the synthesized silica had a desirable spherical structure with a diameter of approximately 100 nm and desirable dispersion.

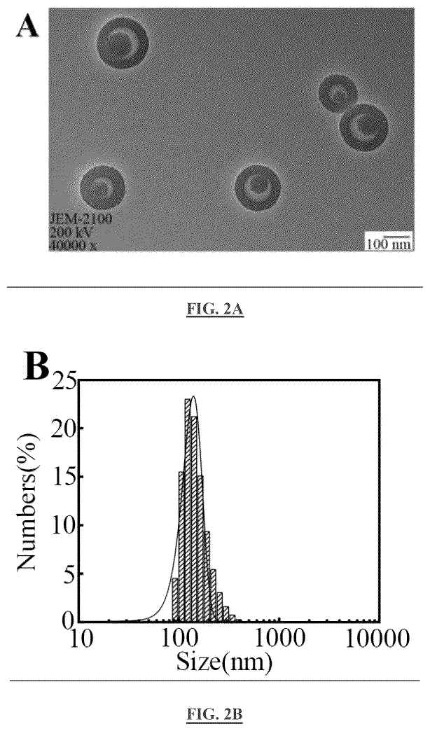

FIGS. 2A-2B showed a TEM image (FIG. 2A) and a DLS data (FIG. 2B) of the HMSN. In FIGS. 2A-B, the TEM data and EDS showed that the HMSN had a desirable spherical structure with a diameter of approximately 150 nm.

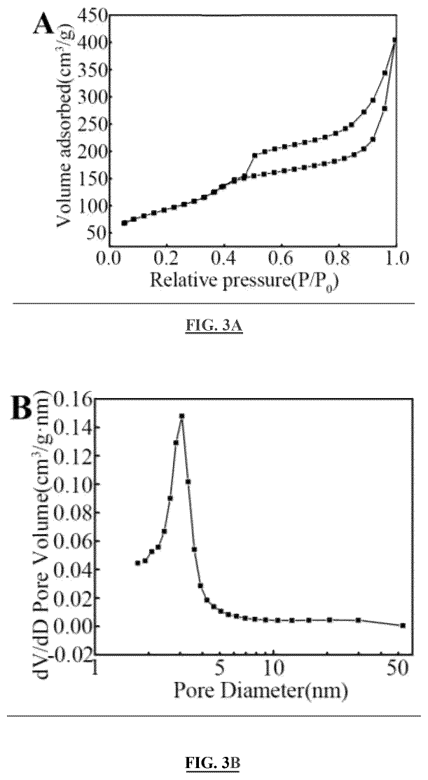

FIGS. 3A-3B showed a nitrogen adsorption-desorption isotherm (FIG. 3A) and pore diameter distribution (FIG. 3B) of the HMSN. In FIGS. 3A-B, the nitrogen adsorption-desorption data illustrated that HMSN had a pore diameter of approximately 3 nm.

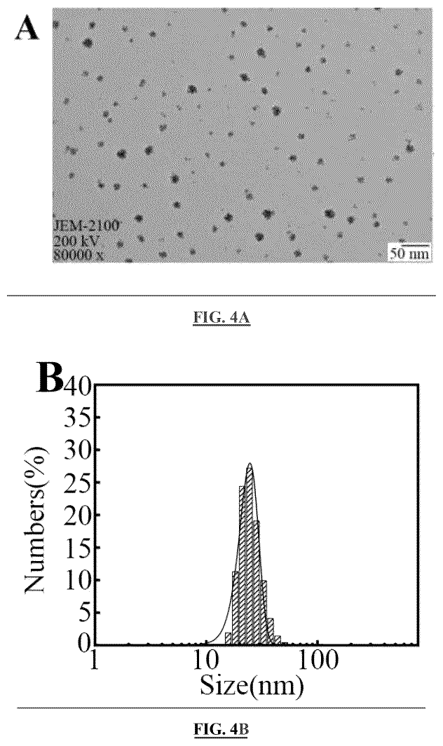

FIGS. 4A-4B shows a TEM image (FIG. 4A) and a DLS data (FIG. 4B) of the positively-charged PtNPs. The data in FIG. 4 showed that the PtNPs had a particle size of approximately 25 nm and desirable dispersion.

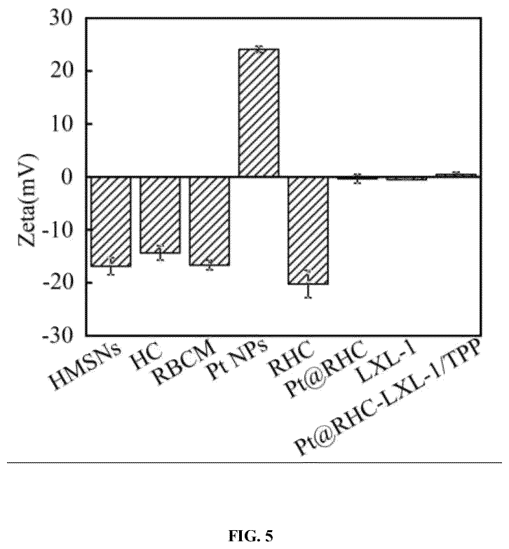

FIG. 5 showed the zeta potentials of HMSN, HC, RBCM, PtNPs, RHC, Pt@RHC, LXL-1, and Pt@RHC-LXL-1/TPP. The zeta potential results in FIG. 5 showed that the HMSN had a negative zeta potential of −16.87 in aqueous solution; after loading it with Ce6, the potential of the material showed −14.37, with no obvious change. RBCM had a negative potential, the Zeta potential was −16.67; after the RBCM coated the Ce6-loaded nanoparticles, the potential was still negative at −20.23. The positively-charged Pt nanocluster had a potential value of 24.1; after the positively-charged Pt nanocluster modified the RHC nanoparticles, the Zeta value changed significantly to −0.35, indicating the successful modification of the material by Pt. TPP had a positive potential, and the aptamer LXL-1 had a potential of −0.519; after asymmetric modification of Pt@RHC, the potential value changed to 0.48, indicating that the modification was successful.

The above results showed that the RHC was synthesized based on the synthesis of HMSN nanoparticles, the Pt@RHC-LXL-1/TPP was obtained by modifying its surface, and then the Pt@RHC-LXL-1/TPP nanoparticle was successfully synthesized.

### 2. Fluorescence Detection

Fluorescence measurements were conducted on the HMSN-coated Ce6 sample (HC) obtained in the fourth step of step 1 of Example 1 to study the photodynamic properties of Ce6 nanoparticles:

Each sample to be tested was diluted with PBS buffer to a final sample concentration of 10 μg/mL for subsequent detection. A fluorescence spectrum from 600 nm to 700 nm was then recorded using an F4600 fluorescence spectrophotometer, with a xenon lamp as the excitation source and 404 nm as the excitation wavelength.

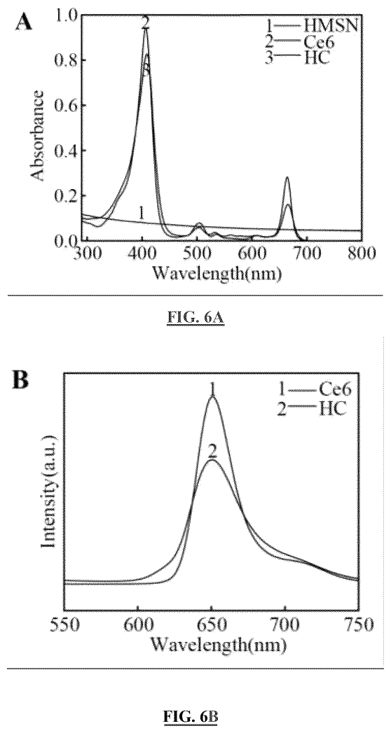

The ultraviolet absorption peaks of Ce6, HMSN, and HMSN-loaded Ce6 (HC) were measured using a UV spectrophotometer, and the fluorescence spectra of Ce6 and HMSN-loaded Ce6 (HC) were measured using a fluorescence spectrometer. The results were shown in FIGS. 6A-6B.

FIG. 6A was the UV absorption curve of HMSN, Ce6, and HC. The results showed that the UV absorption peak of HC was significantly lower than that of Ce6. This was because after HMSN was loaded with Ce6, the fluorescence signal of Ce6 was quenched, indicating that HMSN had been successfully loaded with Ce6. FIG. 6B was the fluorescence intensities of Ce6 and HC. The results showed that the fluorescence spectrum of HC was lower than that of Ce6. This showed that after loading Ce6, the fluorescence intensity of HMSN decreased, indicating that there was still photoresponsiveness after successful loading.

### 3. Detection of Singlet Oxygen in Nanoparticles

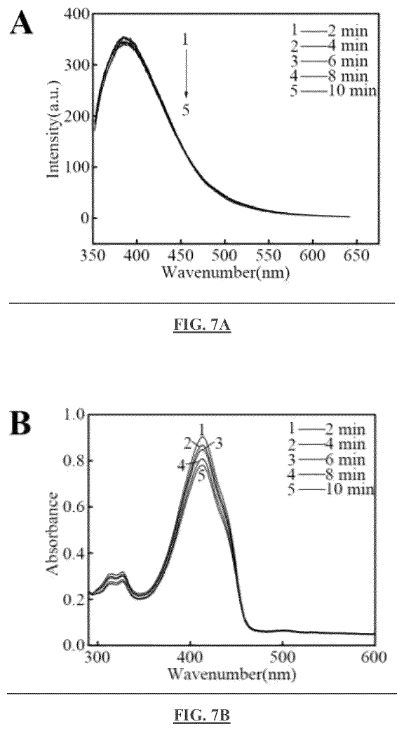

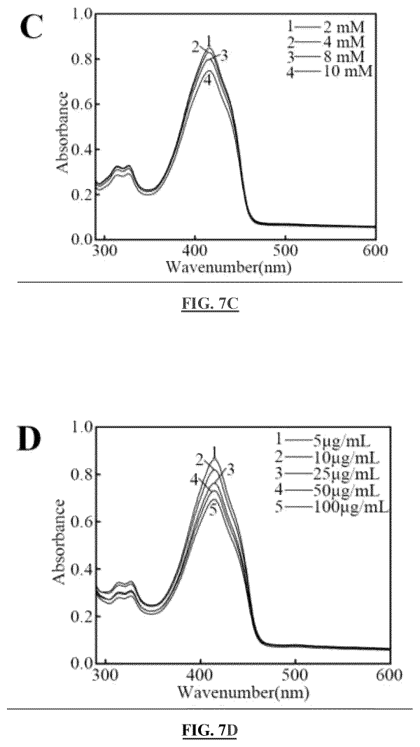

The content of ·OH was determined by the fluorescence intensity of 7-hydroxycoumarin, the product of coumarin. The coumarin (4 mM), H₂O₂ (8 mM), and Pt@RHC-LXL-1/TPP with a concentration of 100 μg/mL were thoroughly mixed, incubated together for 1 h, irradiated for 10 min, the fluorescence curve of the solution was measured every 2 min, and the changes were observed in fluorescence intensity at 455 nm. The results were shown in FIG. 7A.

The Pt@RHC-LXL-1/TPP (100 μg/mL), H₂O₂ (8 mM), and DPBF (20 μM) were incubated at room temperature for 10 min, and irradiated with 808 nm laser for different times (2 min to 10 min), and the absorption curve was recorded. The results were shown in FIG. 7B.

H₂O₂ with different concentrations (2, 4, 8, 10 mM) were separately added into 100 μg/mL Pt@RHC-LXL-1/TPP, incubated with 20 PM DPBF, irradiated with 808 nm laser for 10 min, and the changes of light absorption intensity at 410 nm to 420 nm were observed. The results were shown in FIG. 7C.

In order to evaluate the level of ¹O₂ produced by Pt@RHC-LXL-1/TPP nanoparticles at different concentrations, Pt@RHC-LXL-1/TPP nanoparticles at different concentrations (5, 10, 20, 50, 100 μg/mL) was added with H₂O₂ (8 mM) and 20 μM DPBF, and then irradiated with an 808 nm laser for 10 min, and the changes in light absorption intensity at 410 nm to 420 nm were observed. The results were shown in FIG. 7D.

FIG. 7A showed that there was no obvious fluorescence intensity change at 455 nm, from which it was concluded that no ·OH was produced in this solution. As shown in FIG. 7B, as the irradiation time increased, the absorption peak at 410 nm to 420 nm continued to decrease, indicating that ¹O₂ was produced under this condition. FIG. 7C showed that as the concentration of H₂O₂ increased, the light absorption intensity at 410 nm to 420 nm weakened, that is, the characteristic absorption peak of DPBF gradually decreased, indicating that DPBF was oxidized by ¹O₂. This meant that the concentration of H₂O₂ was positively related to the amount of ¹O₂ produced. FIG. 7D showed that as the concentration of Pt@RHC-LXL-1/TPP increased, the UV absorption peak of DPBF decreased more, indicating that the production of ¹O₂ was affected by the concentration of Pt@RHC-LXL-1/TPP.

### 4. MTT Method in Determining the Cytotoxicity of Nanoparticles

Breast cancer cell MDA-MB-231 was inoculated into a 96-well plate and cultured for 24 h. The experimental groups were set up: 0, 5, 10, 20, 50, 100 μg/mL HMSN were added separately; 0, 5, 10, 20, 50, and 100 μg/mL Pt@RHC were added separately; 0, 5, 10, 20, 50, 100 μg/mL Pt@RHC-LXL-1 were added separately; 0, 5, 10, 20, 50, 100 μg/mL Pt@RHC-LXL-1/TPP were added separately; 0, 5, 10, 20, 50, 100 μg/mL Pt@HC-LXL-1/TPP were added separately; each group was incubated in a cell culture incubator for 12 h, irradiated with 808 nm laser for 10 min, and then incubated for 12 h. An experimental group without illumination was set up: 0, 5, 10, 20, 50, and 100 μg/mL Pt@RHC-LXL-1/TPP were added separately, and incubated in a cell culture incubator for 12 h.

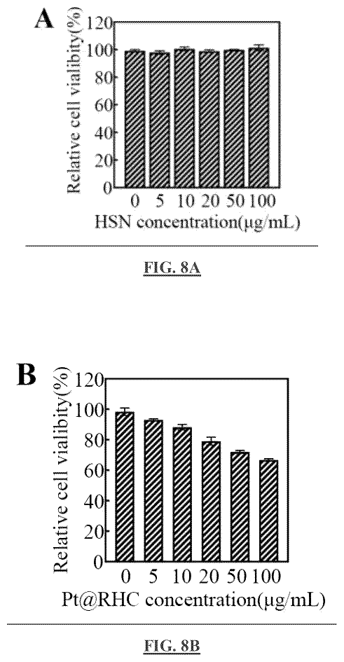

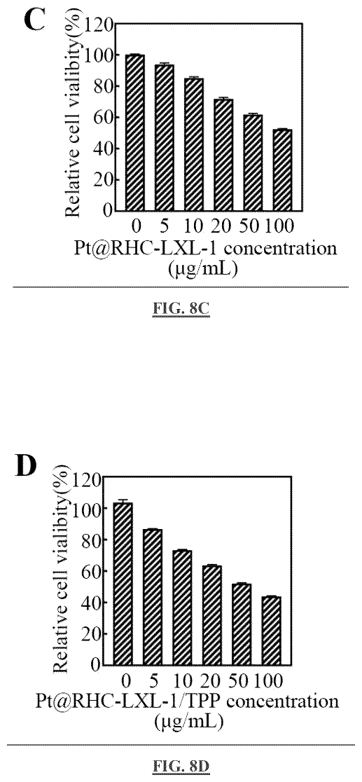

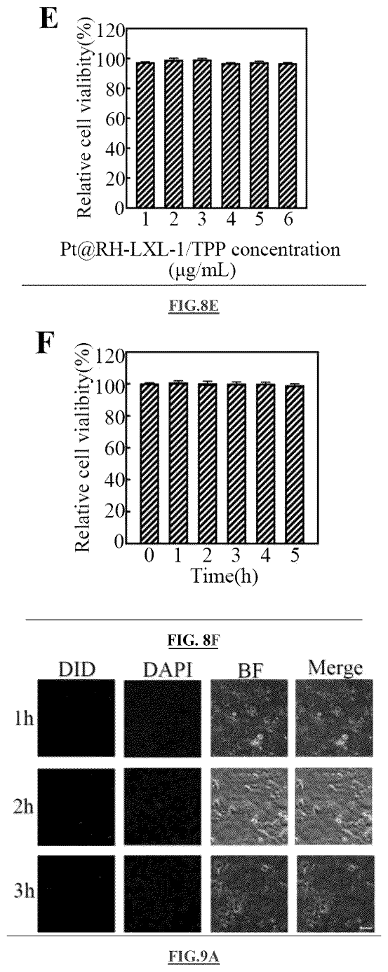

The supernatant was carefully aspirated from each well, 10 μL MTT and 90 μL fresh medium were added into each well, and allowed to stand at 37° C. in the dark for 4 h. After 4 h, the supernatant was aspirated, the remaining substance added with 100 μL of DMSO, oscillated at low speed for 10 min on a oscillator, detected at 490 nm with a microplate reader, and a cell viability was calculated based on the detection data. The results were shown in FIG. 8.

FIG. 8A showed the cell viability of MDA-MB-231 cells co-incubated with HMSN with treatment of illumination. As the concentration of HMSN increased, the cell viability became better, indicating that the HMSN had better biological safety.

FIG. 8B showed the cell viability of MDA-MB-231 cells co-incubated with Pt@RHC with treatment of illumination. As the concentration of Pt@RHC increased, the cytotoxicity of Pt@RHC increased after irradiation with 808 nm laser compared with that of HMSN. This indicated that after Pt@RHC was irradiated by 808 nm laser, Ce6 acted as a photosensitizer to produce a certain amount of ¹O₂, thus damaging mitochondria of breast cancer cells to cause the death of breast cancer cells.

FIG. 8C showed the cell viability of MDA-MB-231 cells co-incubated with Pt@RHC-LXL-1 with treatment of illumination. Comparing the Pt@RHC-LXL-1 and the Pt@RHC of same concentration, the Pt@RHC-LXL-1 showed further increased cytotoxicity. This was because LXL-1 targeted MDA-MB-231, and more Pt@RHC-LXL-1 entered MDA-MB-231 breast cancer cells. More Ce6 could produce more ¹O₂ under light conditions, thereby further increasing cell toxicity.

FIG. 8D showed the cell viability of MDA-MB-231 cells co-incubated with Pt@RHC-LXL-1/TPP with treatment of illumination. Comparing the Pt@RHC-LXL-1/TPP and Pt@RHC-LXL-1 of the same concentration, the Pt@RHC-LXL-1/TPP showed more cytotoxicity after irradiation with 808 nm laser. This was because TPP had the ability to target mitochondria in breast cancer cell MDA-MB-231. Under light conditions, the ¹O₂ produced by Ce6 further increased, causing greater damages to mitochondria DNA and causing greater cell death.

FIG. 8E showed the cell viability of MDA-MB-231 cells co-incubated with Pt@RH-LXL-1/TPP with treatment of illumination. Under light conditions, after comparing Pt@RHC-LXL-1/TPP and Pt@RH-LXL-1/TPP of the same concentration, Pt@RH-LXL-1/TPP showed less cytotoxicity. This was because Pt@RH-LXL-1/TPP did not contain Ce6, and ¹O₂ was not produced even under light conditions. This did not cause mitochondria damage in breast cancer cell MDA-MB-231, so it has less cytotoxic to the cells.

FIG. 8F showed the cell viability of MDA-MB-231 cells co-incubated with Pt@RHC-LXL-1/TPP without illumination. The cell viability of Pt@RHC-LXL-1/TPP nanoparticles basically did not change over time under conditions without light. This indicated that Ce6 could produce ¹O₂ only under light conditions, causing mitochondria damage in cells and further causing cell death.

### 5. Fluorescence Microscopy in Detecting the Targeting of Nanoparticles

In order to study the targeting of Pt@RH-LXL-1/TPP nanoparticles to breast cancer cell MDA-MB-231, breast cancer cell MCF-7 was used as a control experimental group. The breast cancer cell MDA-MB-231 and breast cancer cell MCF-7 were inoculated and cultured for 24 h for targeting analysis.

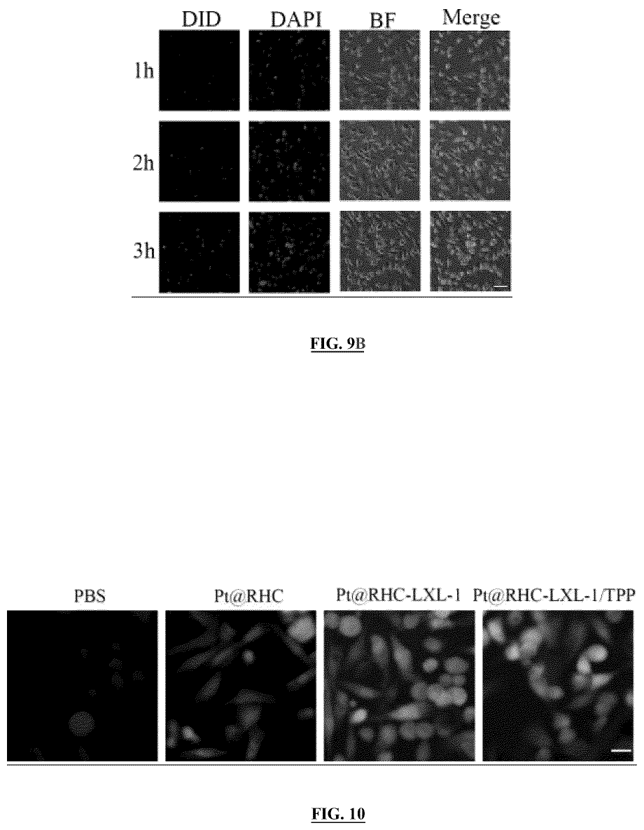

100 μg of DID was incubated overnight with 20 mg of Pt@RH-LXL-1/TPP nanoparticles, where DID had a red fluorescence signal. DID-labeled Pt@RHC-LXL-1/TPP nanoparticles were added into the breast cancer cell MDA-MB-231 and breast cancer cell MCF-7 and co-cultured for 3 h separately. DAPI staining and fluorescence microscope observation of fluorescence intensity were conducted, and the results were shown in FIG. 9.

FIG. 9A was the fluorescence images of breast cancer cell MCF-7 after incubation with the Pt@RH-LXL-1/TPP nanoparticle for 1 h, 2 h, and 3 h, respectively; and FIG. 9B was the fluorescence images of breast cancer cell MDA-MB-231 after incubation with the Pt@RH-LXL-1/TPP nanoparticle for 1 h, 2 h, and 3 h, respectively. Comparing FIG. 9A and FIG. 9B, it was seen that the red fluorescence intensity of breast cancer cell MDA-MB-231 was significantly stronger than that of breast cancer cell MCF-7 during the same time period. Experimental data showed that significantly more Pt@RH-LXL-1/TPP nanoparticles entered breast cancer cell MDA-MB-231 than breast cancer cell MCF-7. The results indicated that Pt@RH-LXL-1/TPP nanoparticles had targeting ability to breast cancer cell MDA-MB-231.

6. Fluorescence Microscopy in Detecting the Production of ¹O₂ in Cells by Nanoparticles

Breast cancer cell MDA-MB-231 was inoculated and cultured for 24 h for detection and analysis of intracellular reactive oxygen species. The PBS solution, 50 μg/mL Pt@RHC nanoparticles, 50 μg/mL Pt@RH-LXL-1 nanoparticles, and 50 μg/mL Pt@RH-LXL-1/TPP nanoparticles were incubated with breast cancer cell MDA-MB-231 for 3 h separately, and irradiated with 808 nm laser for 10 min. The intracellular production of ¹O₂ was detected with the DCFH-DA kit, and the stronger the green fluorescence intensity, the more ¹O₂ was produced. The results were shown in FIG. 10.

FIG. 10 showed that after 808 nm laser irradiation, the green fluorescence intensity of Pt@RHC was stronger than that of the PBS group. This indicated that the nanoparticles entered the cells, Ce6 generated reactive oxygen species under light conditions, and DCFH-DA generated green fluorescence. Compared with Pt@RHC, the fluorescence intensity of Pt@RHC-LXL-1 was further enhanced. This indicated that the targeting of LXL-1 to breast cancer cell MDA-MB-231 allowed more Pt@RHC-LXL-1 nanoparticles to enter breast cancer cell MDA-MB-231, and the ¹O₂ produced after 808 nm laser irradiation also further increased. Comparing the Pt@RHC-LXL-1/TPP and Pt@RHC-LXL-1 groups, it was found that the fluorescence intensity was equivalent. This was because TPP had no effect on the production of ¹O₂ in cells. It was finally proved that when Pt@RHC-LXL-1/TPP nanoparticles were co-incubated with cells, ¹O₂ would be produced in the cells after 808 nm laser irradiation.

In summary, it can be seen that the Pt@RH-LXL-1/TPP nanoparticles prepared by the present disclosure can successfully enter the cells through the special targeting of the aptamer LXL-1 to breast cancer cell MDA-MB-231. Using the targeting of TPP to mitochondria, the Ce6 leaks into mitochondria and cytoplasm, and generates ¹O₂ under irradiation of 808 nm laser to damage the mitochondria and release DNA, causing cell damage and finally cell death.

The research work in the present disclosure is funded by the National Natural Science Foundation of China (project number: 22276102), project title: Nanomotors based on targeted motion for antifungal analysis in the environment and organelle imaging research.

The above descriptions are merely preferred embodiments of the present disclosure. It should be noted that those of ordinary skill in the art may further make several improvements and modifications without departing from the principle of the present disclosure, and such improvements and modifications should be deemed as falling within the protection scope of the present disclosure.

What is claimed is:

1. A photosensitizer chlorin e6 (Ce6)-loaded nanoparticle targeting mitochondria of a breast cancer cell, wherein the nanoparticle uses a hollow mesoporous silica nanoparticle (HMSN) as a carrier, a hollow position of the carrier is loaded with a photosensitizer Ce6, the carrier is externally encapsulated with a red blood cell membrane (RBCM), and the carrier after encapsulating the RBCM is modified with a Pt nanocluster on one side as well as an aptamer LXL-1 and 1-carboxypropyltriphenylphosphine bromide (TPP) on the other side.

2. The nanoparticle according to claim 1, wherein the RBCM is a mouse RBCM.

3. The nanoparticle according to claim 1, wherein the aptamer LXL-1 has the nucleotide sequence set forth in SEQ ID NO: 1.

4. The nanoparticle according to claim 3, wherein a 5′-end of the aptamer LXL-1 is modified with a carboxyl group.

5. A method for preparing the nanoparticle according to claim 1, comprising the following steps:
(1) subjecting the HMSN to ultrasonic mixing with a dimethyl sulfoxide (DMSO) solution of Ce6 to allow freeze-drying to obtain a Ce6-loaded HMSN nanoparticle that is denoted as an HC nanoparticle;
(2) mixing a phosphate-buffered saline (PBS) solution of the HC nanoparticle with a PBS solution of an RBCM powder to allow filter membrane extrusion to obtain an RBCM-coated HC nanoparticle that is denoted as an RHC nanoparticle;
(3) dispersing the RHC nanoparticle in the PBS solution, conducting a first centrifugation, spin-coating a resulting precipitate to a surface of poly-L-lysine, conducting an incubation at room-temperature, washing with the PBS solution, and removing a resulting supernatant; adding a positively-charged platinum (Pt) nanocluster solution into a remaining substance, conducting an incubation at room-temperature, washing with the PBS solution, and removing a resulting supernatant to obtain an asymmetrically-modified nanoparticle that is denoted as a Pt@RHC nanoparticle; and
(4) dispersing the PT@RHC nanoparticle in a PBS solution containing LXL-1, TPP—COOH, 1-ethyl-3-(3-dimethylaminopropyl) carbodiimide hydrochloride (EDC), and N-hydroxysulfosuccinimide sodium salt (NHS) to allow a reaction, stirring at a room temperature, conducting a second centrifugation, washing a resulting precipitate with the PBS solution, and re-dispersing the precipitate in the PBS solution to obtain a modified nanoparticle that is denoted as a Pt@RHC-LXL-1/TPP nanoparticle.

6. The method according to claim 5, wherein a method for preparing the HMSN in step (1) comprises the following steps: adding solid silica into a high-purity aqueous solution of cetyltrimethylammonium chloride (CTAC) and triethanolamine (TEA) to obtain a mixed solution; adding a tetraethyl orthosilicate (TEOS) solution into the mixed solution and stirring in water bath to form a silica surface-coated silica nanoparticle; and etching the silica surface-coated silica nanoparticle to obtain the HMSN.

7. (canceled)

8. (canceled)

9. (canceled)

10. (canceled)

11. (canceled)

12. (canceled)

13. (canceled)

14. (canceled)

15. The method according to claim 5, wherein the HC nanoparticle is washed with ultrapure water.

16. The method according to claim 5, wherein the HC nanoparticle and the RBCM powder in step (2) are at a mass ratio of 1:1.

17. The method according to claim 5, wherein a method for preparing the RBCM powder comprises the following steps: adding water into a red blood cell suspension, allowing to stand such that red blood cells absorb water and burst, conducting centrifugation to remove hemoglobin, washing and dispersing a resulting precipitate with a PBS solution, and freeze-drying to obtain the RBCM powder.

18. (canceled)

19. The method according to claim 5, wherein the filter membrane extrusion in step (2) comprises: conducting extrusion with 450 nm and 200 nm filter membranes in sequence, with each extrusion 4 to 5 times.

20. The method according to claim 5, wherein the first and second centrifugation are conducted at 900 rpm for 3 min and the incubation at room-temperature is conducted for 50 min to 70 min in step (3).

21. The method according to claim 5, wherein a method for preparing the positively-charged Pt nanocluster solution comprises the following steps: adding an H₂PtCl₆ aqueous solution into ultrapure water, adding a protamine aqueous solution, then conducting violent stirring at a room temperature for 30 min, adding NaBH₄ dropwise, stirring a resulting mixture continuously at a room temperature, and conducting dialysis for 24 h to obtain the positively-charged Pt nanocluster solution.

22. The method according to claim 21, wherein the H₂PtCl₆ aqueous solution has a concentration of 16 mM, the protamine aqueous solution has a concentration of 5 mg/mL, and the NaBH₄ has a concentration of 500 mM; and the H₂PtCl₆ aqueous solution, the ultrapure water, the protamine aqueous solution, and the NaBH₄ are at a volume ratio of 0.8:40:0.5:0.4.

23. The method according to claim 5, wherein the TPP—COOH has a concentration of 0.05 g/L to 0.15 g/L, the EDC has a concentration of 0.7 g/L to 0.9 g/L, the NHS has a concentration of 0.3 g/L to 0.5 g/L, and the LXL-1 has a concentration of 100 nM in step (4).

24. The method according to claim 5, wherein the reaction is conducted for 12 min to 18 min, the stirring at the room temperature is conducted for 11 h to 13 h, and the centrifugation is conducted at 12,000 rpm for 15 min in step (4).

25. A method for treating breast cancer wherein comprising using the nanoparticle according to claim 1 combined with illumination.


SEQUENCE LISTING (1 sequence)

SEQ ID NO: 1 - DNA, 83 bp, synthetic construct
  source 1..83: mol_type = other DNA; note = Aptamer LXL-1
gaattcagtcggacagcgaagtagttttccttctaacctaagaacccgcggcagtttaatgtagatggacgaatacgtctccc